(19) United States
(12) Patent Application Publication
(10) Pub. No.: US 2020/0245875 A1
(43) Pub. Date: Aug. 6, 2020

(54) HEARTBEAT DETECTION DEVICE, HEARTBEAT DETECTION METHOD, AND PROGRAM

(71) Applicant: Murakami Corporation, Shizuoka (JP)

(72) Inventor: Atsushi HAYAMI, Shizuoka (JP)

(73) Assignee: Murakami Corporation, Shizuoka (JP)

(21) Appl. No.: 16/641,882

(22) PCT Filed: Aug. 22, 2018
(86) PCT No.: PCT/JP2018/030991

§ 371 Date: Feb. 25, 2020

(30) Foreign Application Priority Data
Sep. 6, 2017 (JP) 2017-170913

(51) Int. Cl. A61B 5/0245; A61B 5/00

(52) U.S. Cl. CPC A61B 5/0245; A61B 2560/0462; A61B 5/72

(57) ABSTRACT

In order to provide a highly reliable heart rate, this heartbeat detection device includes: a first detection unit that detects heartbeats from a vibration wave of a body surface of a user detected by a sensor; a second detection unit that extracts a vibration wave of heartbeats which are amplitude-modulated with a resonant frequency of a human body from the vibration wave of the body surface of the user, delays the extracted vibration wave by a predetermined period, and detects heartbeats from differences between the vibration wave before delay and the vibration waves after delay; and an output control unit that selects either the heartbeats detected by the first detection unit or the heartbeats detected by the second detection unit and determines and outputs a heart rate on the basis of the selected heartbeats.

### TECHNICAL FIELD

The present invention relates to a heartbeat detection device, a heartbeat detection method, and a program.

### BACKGROUND ART

Conventionally, the heartbeats of a driver are detected to provide evidence for determining whether the driver is in a condition suitable for driving. Since a vibration of a body surface of a driver occurs on the body surface due to heartbeats, a pulse wave measurement device that detects the vibration wave of a body surface and calculates a heart rate on the basis of vibration components resulting from the heartbeats extracted from the vibration wave has been proposed (for example, see Patent Literature 1). When a vibration wave occurring on the body surface on the back side of a driver is detected using sensors incorporated into a seat, it is possible to calculate a heart rate without restraining the driver.

### CITATION LIST

### Patent Literature

[Patent Literature 1] Japanese Unexamined Patent Application Publication No. 2016-54814

### SUMMARY OF INVENTION

### Technical Problem

However, large vibration components resulting from the body motion of the driver are likely to be superimposed on the vibration wave of the body surface as noise and the waveform is sometimes disordered greatly. The heart rate calculated from a vibration wave of which the waveform is greatly disordered has a large error from the actual heart rate due to the influence of noise and the reliability thereof may be reduced.

An object of the present invention is to provide a highly reliable heart rate.

### Solution to Problem

According to the invention described in claim 1, there is provided a heartbeat detection device including:

a first detection unit that detects heartbeats from a vibration wave of a body surface of a user detected by a sensor;

a second detection unit that extracts a vibration wave of heartbeats which are amplitude-modulated with a resonant frequency of a human body from the vibration wave of the body surface of the user, delays the extracted vibration wave by a predetermined time, and detects heartbeats from differences between the vibration wave before delay and the vibration waves after delay; and

an output control unit that selects either the heartbeats detected by the first detection unit or the heartbeats detected by the second detection unit, and determines and outputs a heart rate on the basis of the selected heartbeats.

In this way, a heart rate can be determined on the basis of either the heartbeats detected by the first detection unit or the heartbeats detected by the second detection unit. Therefore, a heart rate can be generally output on the basis of the heartbeats detected by the first detection unit, and the heart rate can be output on the basis of the heartbeats detected by the second detection unit when the reliability of the heartbeats detected by the first detection unit decreases due to disorder in the vibration waves of the body surface caused by a body motion of a user. The heartbeats detected by the second detection unit are heartbeats detected by removing vibration components resulting from the body motion of the user from the vibration waves of the body surface and are less influenced by body motion. Therefore, it is possible to provide a highly reliable heart rate with little error resulting from a body motion.

According to the invention described in claim 2, there is provided the heartbeat detection device according to claim 1, further including:

a determination unit that determines a reliability of the heartbeats detected by the first detection unit, wherein

the output control unit selects the heartbeats depending on a determination result obtained by the determination unit.

In this way, the heartbeats detected by the first detection unit or the second detection unit are selected according to the determination result of the reliability of the heartbeats detected by the first detection unit, and the heart rate can be determined on the basis of the selected heartbeats.

According to the invention described in claim 3, there is provided the heartbeat detection device according to claim 2, in which

when the determination unit determines that the reliability of the heartbeats detected by the first detection unit is low, the output control unit selects the heartbeats detected by the second detection unit and determines the heart rate.

In this way, when the reliability of the heartbeats detected by the first detection unit is low, a highly reliable heart rate can be determined on the basis of the heartbeats with little error resulting from a body motion, detected by the second detection unit.

According to the invention described in claim 4, there is provided the heartbeat detection device according to claim 2 or 3, in which

when the determination unit determines that the reliability of the heartbeats detected by the first detection unit is high, the output control unit selects the heartbeats detected by the first detection unit and determines the heart rate.

In this way, when the reliability of the heartbeats detected by the first detection unit is high, the heart rate can be determined on the basis of highly reliable heartbeats.

According to the invention described in claim 5, there is provided the heartbeat detection device according to any one of claims 2 to 4, in which

the second detection unit detects peaks of which the period corresponds to the heartbeats which have been detected by the first detection unit and of which the reliability is determined to be high by the determination unit as the heartbeats among a plurality of peaks in which the difference is smaller in waveforms of the differences between the vibration waves before and after delay.

In this way, even when vibration wave components having a periodicity other than that of heartbeats are included in the vibration waves before delay, heartbeats can be detected with high accuracy.

According to the invention described in claim 6, there is provided a heartbeat detection method including:

a first detection step of detecting heartbeats from a vibration wave of a body surface of a user detected by a sensor;

a second detection step of extracting a vibration wave of heartbeats which are amplitude-modulated with a resonant frequency of a human body from the vibration wave of the body surface of the user, delaying the extracted vibration wave by a predetermined time, and detecting heartbeats from differences between the vibration wave before delay and the vibration waves after delay; and

an output step of selecting either the heartbeats detected in the first detection step or the heartbeats detected in the second detection step, and determining and outputting a heart rate on the basis of the selected heartbeats.

In this way, a heart rate can be determined on the basis of either the heartbeats detected in the first detection step or the heartbeats detected in the second detection step. Therefore, a heart rate can be generally output on the basis of the heartbeats detected in the first detection step, and the heart rate can be output on the basis of the heartbeats detected in the second detection step when the reliability of the heartbeats detected in the first detection step decreases due to disorder in the vibration waves of the body surface caused by a body motion of a user. The heartbeats detected in the second detection step are heartbeats detected by removing vibration components resulting from the body motion of the user from the vibration waves of the body surface and are less influenced by body motion. Therefore, it is possible to provide a highly reliable heart rate with little error resulting from a body motion.

According to the invention described in claim 7, there is provided a program for causing a computer to execute:

a first detection step of detecting heartbeats from a vibration wave of a body surface of a user detected by a sensor;

a second detection step of extracting a vibration wave of heartbeats which are amplitude-modulated with a resonant frequency of a human body from the vibration wave of the body surface of the user, delaying the extracted vibration wave by a predetermined time, and detecting heartbeats from differences between the vibration wave before delay and the vibration waves after delay; and

an output step of selecting either the heartbeats detected in the first detection step or the heartbeats detected in the second detection step and determining and outputting a heart rate on the basis of the selected heartbeats.

In this way, a heart rate can be determined on the basis of either the heartbeats detected in the first detection step or the heartbeats detected in the second detection step. Therefore, a heart rate can be generally output on the basis of the heartbeats detected in the first detection step, and the heart rate can be output on the basis of the heartbeats detected in the second detection step when the reliability of the heartbeats detected in the first detection step decreases due to disorder in the vibration waves of the body surface caused by a body motion of a user. The heartbeats detected in the second detection step are heartbeats detected by removing vibration components resulting from the body motion of the user from the vibration waves of the body surface and are less influenced by body motion. Therefore, it is possible to provide a highly reliable heart rate with little error resulting from a body motion.

### Advantageous Effects of Invention

According to the present invention, it is possible to provide a highly reliable heart rate.

### BRIEF DESCRIPTION OF DRAWINGS

FIG. 1 is a block diagram illustrating a functional configuration of a heartbeat detection device according to an embodiment of the present invention.

FIG. 2 is a graph illustrating an example of vibration waves extracted from vibration waves of a body surface by a second detection unit.

FIG. 3 is a diagram illustrating an example of the vibration waves extracted from the vibration waves of the body surface and vibration waves obtained by delaying the vibration waves by a predetermined time.

FIG. 4 is a diagram illustrating an example of a waveform of differences between the vibration wave before delay and the vibration waves after delay.

FIG. 5 is a flowchart illustrating a processing order when the heartbeat detection device outputs a heart rate.

FIG. 6 is a graph illustrating an example of heart rates of the heartbeats detected by the first and second detection units.

### DESCRIPTION OF EMBODIMENTS

An embodiment of a heartbeat detection device, a heartbeat detection method, and a program according to the present invention will be described below with reference to the drawings.

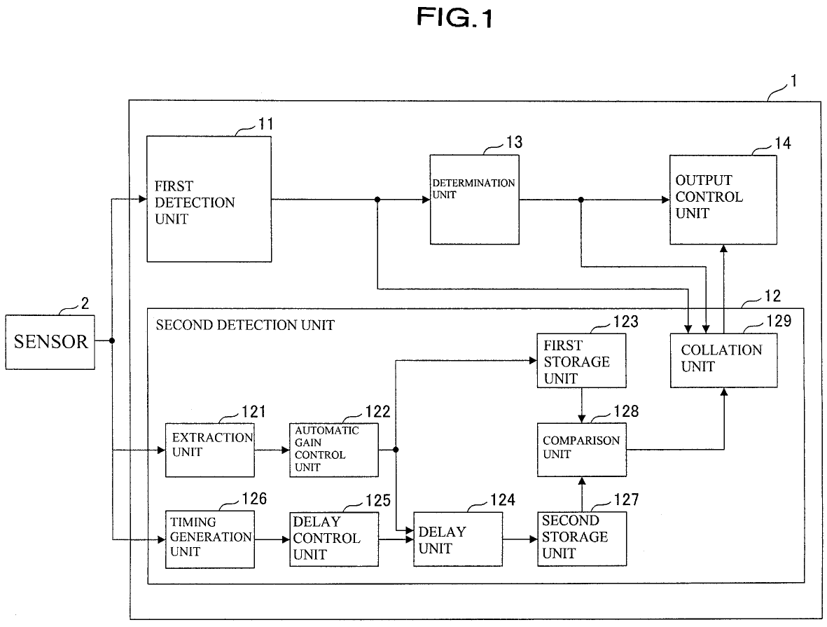

FIG. 1 is a block diagram illustrating a functional configuration of a heartbeat detection device 1 according to an embodiment of the present invention.

As illustrated in FIG. 1, the heartbeat detection device 1 is connected to a sensor 2 to detect heartbeats of a user from the vibration waves of a body surface of the user detected by the sensor 2, and outputs the heart rate.

(Sensor)

The sensor 2 detects vibration waves occurring on the body surface of the user. A microphone sensor or the like, for example, can be used as the sensor 2.

The sensor 2 can detect the vibration waves of the body surface on the back side of the user by being disposed in a pad or the like used in close-contact with the back side of the user as disclosed in Japanese Unexamined Patent Application Publication No. 2016-54814, for example. This pad includes a three-dimensional solid knit that generates a tensile force by receiving a pressure from the back side of the user and propagates vibration of the body surface on the back side to the sensor 2.

(Heartbeat Detection Device)

As illustrated in FIG. 1, the heartbeat detection device 1 includes a first detection unit 11, a second detection unit 12, a determination unit 13, and an output control unit 14.

The details of the processes of the respective configuration units of the heartbeat detection device 1 can be realized by hardware such as a field-programmable gate array (FPGA) or a large scale integration (LSI). Moreover, the details of the processes of the respective configuration units can be also realized by software processing in which a computer reads and executes a program that describes the details of the processes from a storage medium storing the program therein. A processor such as a central processing unit (CPU) or a graphics processing unit (GPU), for example, can be used as the computer. A hard disk, a read only memory (ROM), or the like can be used as the storage medium.

The first detection unit 11 acquires the vibration waves of the body surface of the user detected by the sensor 2 and detects the heartbeats from the vibration waves.

An arbitrary method may be used as a heartbeat detection method in the first detection unit 11 as long as the detection unit can independently detect the heartbeats from the vibration waves of the body surface. For example, a detection method disclosed in Japanese Unexamined Patent Application Publication No. 2016-54814 can be used as the detection method of the first detection unit 11. In this detection method, a reference wave of a frequency band of heartbeats and an emphasis wave including the frequency band of the heartbeats and a higher frequency band are extracted from the vibration waves of the body surface, and the peaks of the emphasis wave appearing near the peaks of the extracted reference wave are detected as heartbeats.

The second detection unit 12 acquires vibration waves of the body surface of the user detected by the sensor 2. Since the vibration waves of the heartbeats in the vibration waves of the body surface are amplitude-modulated with a resonant frequency of a human body, the second detection unit 12 extracts vibration waves of the heartbeats amplitude-modulated with the resonant frequency of a human body from the vibration waves of the body surface. The second detection unit 12 delays the extracted vibration wave by a predetermined time and detects heartbeats from the differences between the vibration wave before delay and the vibration waves after delay.

As illustrated in FIG. 1, the second detection unit 12 includes an extraction unit 121, an automatic gain control unit 122, a first storage unit 123, a delay unit 124, a delay control unit 125, a timing generation unit 126, a second storage unit 127, a comparison unit 128, and a collation unit 129.

The extraction unit 121 extracts vibration waves of the heartbeats amplitude-modulated with the resonant frequency of the human body from the vibration wave of the body surface of the user. Specifically, the extraction unit 121 extracts a certain range of frequency bands around the resonant frequency of the human body. The certain range can be determined by the frequency of the heartbeats, and the extraction unit 121 extracts vibration waves of a frequency band of (resonant frequency of human body)±(frequency of heartbeats), for example. The extraction unit 121 can extract the vibration waves of an intended frequency band by filtering the vibration waves of the body surface using a band-pass filter, a high-pass filter, a low-pass filter or the like.

Although there are individual differences, the frequency of vibration waves of heartbeats is generally near 1 Hz and varies within the range of approximately 0.7 to 2.0 Hz depending on a body condition. On the other hand, there is no specific data of the resonant frequency of a human body, and there are various opinions such as 5 Hz, 8 Hz, 30 Hz, and the like. For example, when the resonant frequency of a human body is 8 Hz, the extraction unit 121 extracts vibration waves of a frequency band in a range of +2 Hz about 8 Hz, that is, 6 to 10 Hz.

A vibration larger than the heartbeats may occur on the body surface of the user due to the body motion of the user, the vibration waves may become noise components and may greatly disorder the waveform, and the output value from the sensor 2 may become saturated. The vibration waves of the body motion are in a frequency band lower than the resonant frequency of the human body, for example, approximately 0.1 to 0.5 Hz. Therefore, as described above, by extracting the vibration waves of the frequency band around the resonant frequency of the human body from the vibration waves of the body surface, it is possible to remove the vibration waves of body motion serving as noise components.

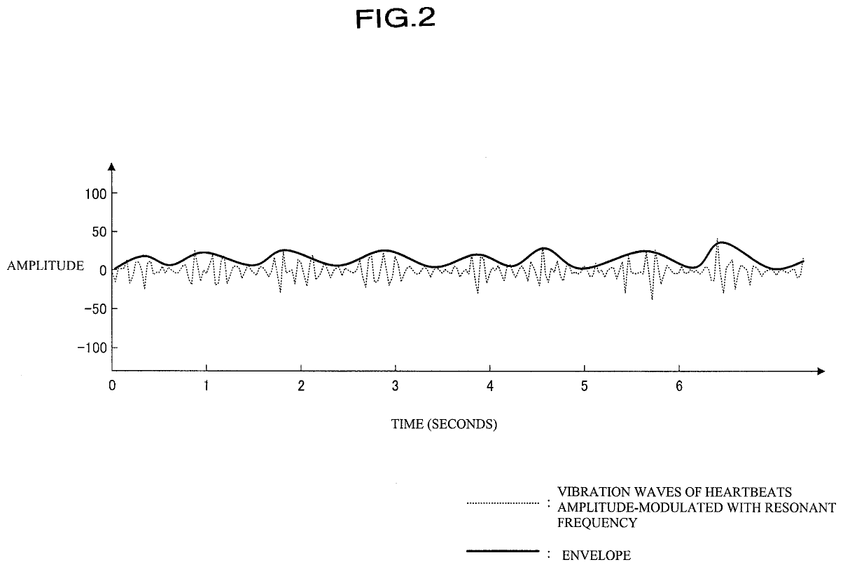

FIG. 2 illustrates an example of vibration waves extracted by the extraction unit 121 from the vibration waves of the body surface.

Since the vibration waves extracted by the extraction unit 121 are vibration waves of the heartbeats amplitude-modulated with the resonant frequency of the human body, it can be said that the envelope of the extracted vibration waves indicates the period of heartbeats as illustrated in FIG. 2.

The automatic gain control unit 122 outputs the vibration waves extracted by the extraction unit 121 by adjusting the amplitude of long period vibration wave components which have no influence on the heartbeats to be constant.

The first storage unit 123 stores the vibration waves output from the automatic gain control unit 122. A buffer memory or the like can be used as the first storage unit 123.

The delay unit 124 delays the vibration waves output from the automatic gain control unit 122 by a predetermined time according to an instruction of the delay control unit 125 and outputs the vibration waves after delay.

The delay control unit 125 instructs the delay unit 124 to delay the vibration waves according to a clock signal generated by the timing generation unit 126.

The timing generation unit 126 generates the clock signal for A/D converting the output of the sensor 2.

The second storage unit 127 stores the vibration waves after delay output from the delay unit 124. A ring buffer memory or the like can be used as the second storage unit 127.

The comparison unit 128 calculates differences between the vibration waves before delay stored in the first storage unit 123 and the vibration waves after delay stored in the second storage unit 127.

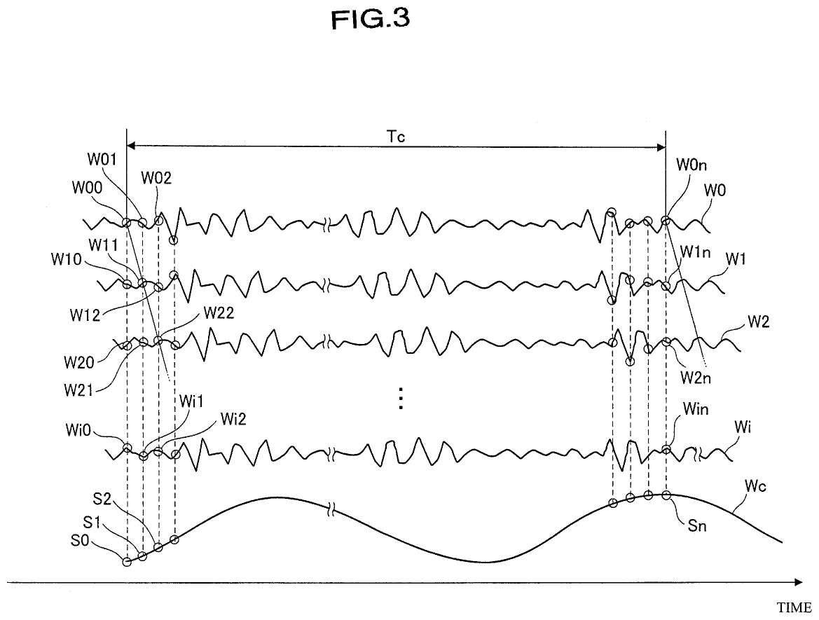

FIG. 3 illustrates an example of the vibration wave before delay and the vibration waves after delay.

As illustrated in FIG. 3, vibration waves Wi delayed by times which are i-times (i is an integer of 1 or more) a predetermined time t from an original vibration wave W0 are obtained by the delay unit 124. For example, a vibration wave W1 is a vibration wave delayed by the predetermined time t from the vibration wave W0, and a vibration wave W2 is a vibration wave further delayed by the predetermined time t from the vibration wave W1, that is, a vibration wave delayed by time 2t from the vibration wave W0.

The comparison unit 128 compares the vibration wave W0 before delay and the vibration waves Wi after delay in a calculation period Tc and calculates the difference.

The calculation period Tc can be determined depending on the period of heartbeats to be detected. For example, when heartbeats of which the heart rate is 30 BPM or more are detected, since at least two seconds are required for detecting one period of heartbeats, the calculation period Tc may be determined to be two seconds or more.

Specifically, the comparison unit 128 samples the vibration wave W0 before delay and the vibration waves Wi after delay at a fixed sampling interval within the calculation period Tc. The sampling interval is the same time as the delay amount of the vibration waves Wi. The comparison unit 128 calculates the sum Sj of the absolute values of the differences between the sampled vibration wave W0j before delay and the sampled vibration waves Wij after delay as illustrated in the following equation. Here, j is a number indicating the number of sampling times, and j is 0 to n.

Sj=Σ{abs(W0j−Wij)}

In the equation, abs( ) indicates a function that outputs an absolute value of a calculation result in the parentheses ( ). W0j indicates an amplitude value of the sampled vibration wave W0 before delay. Wij indicates amplitude values of the sampled vibration waves Wi after delay.

For example, S0, S1, S2, . . . , Sn in FIG. 3 can be calculated as follows.

S0=abs(W00−W00)+abs(W01−W01)++abs(W0n−W0n)

S1=abs(W00−W10)+abs(W01−W11)++abs(W0n−W1n)

S2=abs(W0O−W20)+abs(W01−W21)++abs(W0n−W2n)

. . .

Sn=abs(W0O−Wi0)+abs(W01−Wi1)++abs(W0n−Win)

When a vibration wave having a periodicity like heartbeats is delayed by a predetermined time, although a difference from an original vibration wave increases, the difference becomes the smallest when the period of the vibration wave matches the period of the vibration wave which is further delayed. Therefore, as illustrated in FIG. 3, when Sj is output at the same sampling interval as the delay time, the original vibration wave W0, that is, a vibration wave Wc which is a repetition wave having the fundamental period which is the period of a modulation wave obtained by amplitude-modulating the vibration wave of heartbeats with the resonant frequency of a human body can be obtained. The vibration wave Wc exhibits autocorrelation of the original vibration wave W0, and a higher autocorrelation is obtained as the amplitude value decreases.

The delay unit 124 outputs the delayed vibration waves Wi in the calculation period Tc.

For example, when the delay time of the vibration wave W0 is 1/32 seconds and the calculation period Tc is eight seconds, the delay unit 124 outputs vibration waves W1 to W255. Since the sampling interval is 1/32 seconds that is the same as the delay time, sampling is performed 256 times in the calculation period Tc.

The collation unit 129 collates a plurality of peaks in which the difference becomes the smallest among the waveforms of the differences between the vibration wave before delay and the vibration waves after delay output from the comparison unit 128 with the heartbeats which is detected by the first detection unit 11 and of which the reliability is determined to be high by the determination unit 13. The collation unit 129 uses the heartbeats in collation while the reliability of the heartbeats detected by the first detection unit 11 is determined to be high by the determination unit 13. The collation unit 129 stores heartbeats within a predetermined period determined to be highly reliable from the present time point, and uses the latest heartbeat among those determined to be highly reliable in the collation and stored therein, when the reliability of the heartbeats detected by the first detection unit 11 is determined to be low. In this way, it is possible to output the heartbeats having high reliability by the second detection unit 12.

The collation unit 129 detects peaks of which the period corresponds to the collated heartbeats among the plurality of peaks as the heartbeats. The collation unit 129 can determine that peaks have a period corresponding to the heartbeats detected by the first detection unit 11 when the time difference between the peak and the heartbeats detected by the first detection unit 11 is equal to or smaller than a threshold. The threshold can be set appropriately within an allowable error range.

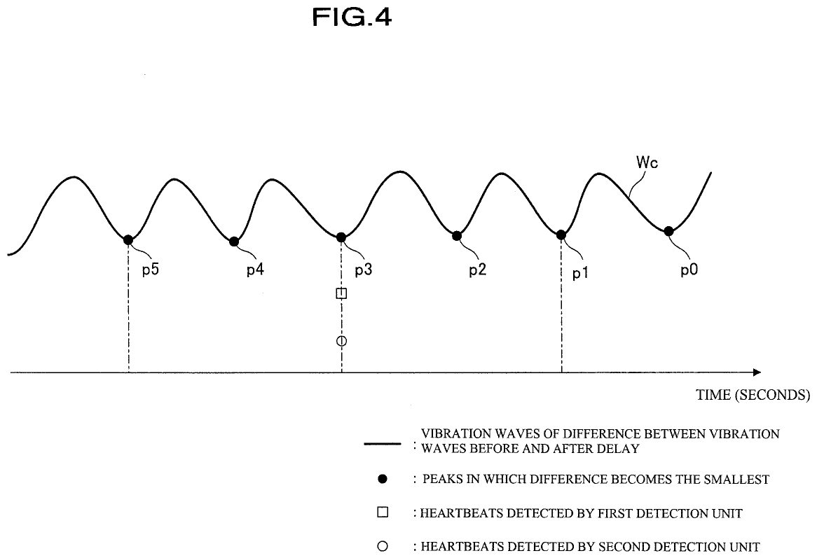

FIG. 4 illustrates an example of a waveform of differences between the vibration waves before and after delay.

As illustrated in FIG. 4, the vibration wave We which is the differences between the vibration waves before and after delay has a plurality of peaks p0 to p5 at which the difference becomes the smallest. When a vibration wave having a periodicity like heartbeats has a same period as a delayed vibration wave, since the difference becomes the smallest, there is a possibility that the peaks p0 to p5 are heartbeats. However, since the vibration wave before delay may contain vibration wave components having a periodicity other than that of heartbeats, it cannot be said that all peaks p0 to p5 are heartbeats. The collation unit 129 detects the peak p3 of which the period corresponds to the heartbeats detected by the first detection unit 11 among the peaks p0 to p5 as heartbeats.

The determination unit 13 determines the reliability of the heartbeats detected by the first detection unit 11. For example, the determination unit 13 calculates a heart rate from the period of the heartbeats detected by the first detection unit 11 and calculates a variance of five latest heart rates that have been calculated. The determination unit 13 can determine that the reliability is low when the variance is equal to or larger than a threshold and can determine that the reliability is high when the variance is smaller than the threshold. Moreover, the determination unit 13 can determine that the reliability is low when the vibration wave is greatly disordered due to the movement of a user and the output of the sensor 2 becomes saturated.

The output control unit 14 selects either the heartbeats detected by the first detection unit 11 or the heartbeats detected by the second detection unit 12, and determines and outputs a heart rate using the selected heartbeats.

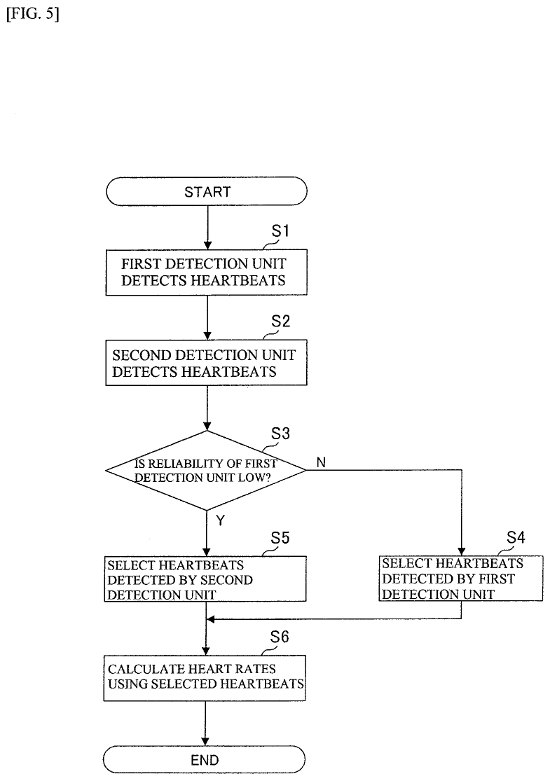

FIG. 5 is a flowchart illustrating a processing order when the heartbeat detection device 1 detects heartbeats.

In the heartbeat detection device 1, as illustrated in FIG. 5, the first detection unit 11 detects heartbeats from the vibration wave of the body surface input from the sensor 2 (step S1). The second detection unit 12 also detects heartbeats from the same vibration wave (step S2). The determination unit 13 determines the reliability of the heartbeats detected by the first detection unit 11 (step S3).

When the determination unit 13 determines that the reliability of heartbeats is high (step S3: N), the output control unit 14 selects the heartbeats detected by the first detection unit 11 among the heartbeats detected by the first detection unit 11 and second detection unit 12 (step S4). The output control unit 14 determines a heart rate using the selected heartbeats and outputs the heart rate (step S6).

The output control unit 14 may calculate the heart rate directly from the period of the heartbeats detected by the first detection unit 11 and may calculate the heart rate by analyzing time-series heartbeats detected by the first detection unit 11. The heart rate calculated by time-series analysis can be obtained by filtering the period of the heartbeats detected by the first detection unit 11 for a predetermined period from the present time point using a Kalman filter, for example. A more accurate heart rate can be provided by time-series analysis.

On the other hand, when the determination unit 13 determines that the reliability of the heartbeats is low (step S3: Y), the output control unit 14 selects the heartbeats detected by the second detection unit 12 among the heartbeats detected by the first detection unit 11 and second detection unit 12 (step S5). The output control unit 14 determines a heart rate using the selected heartbeat and outputs the heart rate (step S6). In this case, similarly, the output control unit 14 may calculate the heart rate directly from the period of the heartbeats detected by the second detection unit 12 and may calculate the heart rate by performing time-series analyze of heartbeats detected by the second detection unit 12.

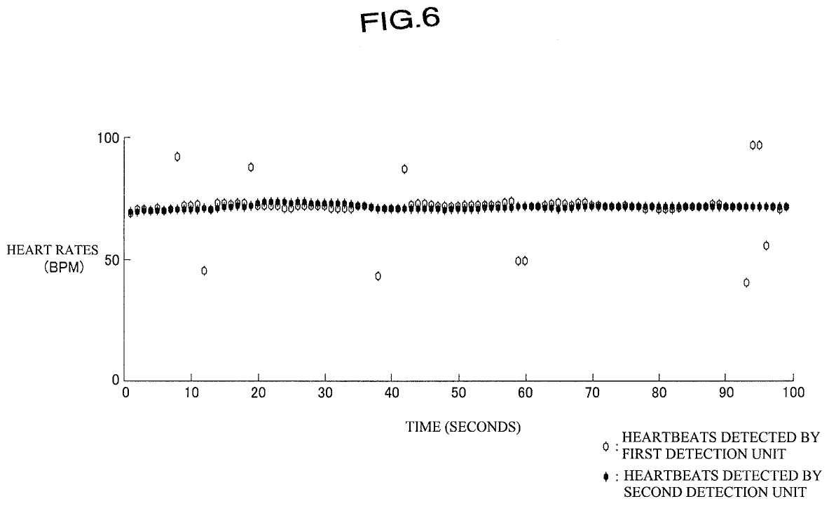

FIG. 6 illustrates an example of the heart rates (BPM) calculated from the heartbeats detected by the first detection unit 11 and second detection unit 12.

As illustrated in FIG. 6, the heart rate (BPM) calculated using the heartbeats of the first detection unit 11 varies greatly due to the influence of the vibration wave of a body motion when the body motion of a user occurs. On the other hand, it is found that the heart rate (BPM) calculated using the heartbeats of the second detection unit 12 varies a little even when body motion occurs and is highly reliable.

As illustrated in FIG. 6, it is possible to provide a heart rate that is always highly reliable by calculating the heart rate using the heartbeats of the first detection unit 11 while the reliability of the heartbeats of the first detection unit 11 is high, and using the heartbeats of the second detection unit 12 while the reliability of the heartbeats of the first detection unit 11 is low.

As described above, the heartbeat detection device 1 of the present embodiment includes: the first detection unit 11 that detects heartbeats from a vibration wave of a body surface of a user detected by the sensor 2; the second detection unit 12 that extracts a vibration wave of heartbeats which are amplitude-modulated with a resonant frequency of a human body from the vibration wave of the body surface of the user, delays the extracted vibration wave by a predetermined time, and detects heartbeats from differences between the vibration wave before delay and the vibration waves after delay; and the output control unit 14 that selects either the heartbeats detected by the first detection unit 11 or the heartbeats detected by the second detection unit 12 and determines and outputs a heart rate on the basis of the selected heartbeats.

According to the embodiment, a heart rate can be determined on the basis of either the heartbeats detected by the first detection unit 11 or the heartbeats detected by the second detection unit 12. A heart rate can be output on the basis of the heartbeats detected by the first detection unit 11 when the reliability of the heartbeats detected by the first detection unit 11 is high, and the heart rate can be output on the basis of the heartbeats detected by the second detection unit 12 when the reliability of the heartbeats detected by the first detection unit 11 is low due to disorder in the vibration waves of the body surface caused by a body motion of a user. The heartbeats detected by the second detection unit 12 are heartbeats detected by removing vibration components resulting from the body motion of the user from the vibration waves of the body surface and are less influenced by body motion. Therefore, it is possible to provide a highly reliable heart rate with little error resulting from a body motion.

The embodiment is a preferred example of the present invention and the present invention is not limited thereto. The embodiment can be changed appropriately within the range of the technical idea of the present invention.

For example, in the embodiment, the first detection unit 11 and second detection unit 12 acquire a vibration wave of the body surface from the same sensor 2. However, the first detection unit 11 and second detection unit 12 may acquire vibration waves from different sensors as long as it is possible to acquire a vibration wave of the body surface of the same user.

In the processing order, the output control unit 14 selects the heartbeats detected by the first detection unit 11 or second detection unit 12 and determines a heart rate using the selected heartbeats only. The present invention is not limited thereto, and the heart rate may be determined using non-selected heartbeats supplementally to the selected heartbeats if the heart rate is determined on the basis of the selected heartbeats.

Specifically, the output control unit 14 interpolates the heartbeats of the first detection unit 11 and second detection unit 12 by weighting the selected heartbeats and determines a heart rate from the interpolated heartbeats. For example, when the heartbeats of the second detection unit 12 are selected, the output control unit 14 interpolates the heartbeats by weighting the heartbeats of the second detection unit 12 with a weight of 9/10 and the heartbeats of the first detection unit 11 with a weight of 1/10 and determines the heart rate from the obtained heartbeats.

In the processing order, the output control unit 14 selects the heartbeats according to the determination result obtained by the determination unit 13 and determines the heart rate. The present invention is not limited thereto, the output control unit 14 may always determine the heart rate on the basis of the selected heartbeats regardless of the determination result of the determination unit 13. For example, the output control unit 14 may generally select the heartbeats of the first detection unit 11 and may select the heartbeats of the second detection unit 12 when a large movement of the user exceeding a threshold is detected by a sensor such as an acceleration sensor.

This application claims priority to and the benefit from Japanese Patent Application No. 2017-170913, filed on Sep. 6, 2017, the contents of which are hereby incorporated by reference into the present application.

### REFERENCE SIGNS LIST

1 Heartbeat detection device
11 First detection unit
12 Second detection unit
121 Extraction unit
123 First storage unit
124 Delay unit
127 Second storage unit
128 Comparison unit
129 Collation unit
13 Determination unit
14 Output control unit
2 Sensor

1. A heartbeat detection device comprising:
a first detection unit that detects heartbeats from a vibration wave of a body surface of a user detected by a sensor;
a second detection unit that extracts a vibration wave of heartbeats which are amplitude-modulated with a resonant frequency of a human body from the vibration wave of the body surface of the user, delays the extracted vibration wave by a predetermined time, and detects heartbeats from differences between the vibration wave before delay and the vibration waves after delay; and
an output control unit that selects either the heartbeats detected by the first detection unit or the heartbeats detected by the second detection unit and determines and outputs a heart rate on the basis of the selected heartbeats.

2. The heartbeat detection device according to claim 1, further comprising:
a determination unit that determines a reliability of the heartbeats detected by the first detection unit, wherein
the output control unit selects the heartbeats depending on a determination result obtained by the determination unit.

3. The heartbeat detection device according to claim 2, wherein
when the determination unit determines that the reliability of the heartbeats detected by the first detection unit is low, the output control unit selects the heartbeats detected by the second detection unit and determines the heart rate.

4. The heartbeat detection device according to claim 2, wherein
when the determination unit determines that the reliability of the heartbeats detected by the first detection unit is high, the output control unit selects the heartbeats detected by the first detection unit and determines the heart rate.

5. The heartbeat detection device according to claim 2, wherein
the second detection unit detects peaks of which the period corresponds to the heartbeats which is detected by the first detection unit and of which the reliability is determined to be high by the determination unit as the heartbeats among a plurality of peaks in which the difference becomes the smallest in waveforms of the differences between the vibration waves before and after delay.

6. A heartbeat detection method comprising:
a first detection step of detecting heartbeats from a vibration wave of a body surface of a user detected by a sensor;
a second detection step of extracting a vibration wave of heartbeats which are amplitude-modulated with a resonant frequency of a human body from the vibration wave of the body surface of the user, delaying the extracted vibration wave by a predetermined time, and detecting heartbeats from differences between the vibration wave before delay and the vibration waves after delay; and
an output step of selecting either the heartbeats detected in the first detection step or the heartbeats detected in the second detection step, and determining and outputting a heart rate on the basis of the selected heartbeats.

7. A program for causing a computer to execute:
a first detection step of detecting heartbeats from a vibration wave of a body surface of a user detected by a sensor;
a second detection step of extracting a vibration wave of heartbeats which are amplitude-modulated with a resonant frequency of a human body from the vibration wave of the body surface of the user, delaying the extracted vibration wave by a predetermined time, and detecting heartbeats from differences between the vibration wave before delay and the vibration waves after delay; and
an output step of selecting either the heartbeats detected in the first detection step or the heartbeats detected in the second detection step, and determining and outputting a heart rate on the basis of the selected heartbeats.

8. The heartbeat detection device according to claim 3, wherein
the second detection unit detects peaks of which the period corresponds to the heartbeats which is detected by the first detection unit and of which the reliability is determined to be high by the determination unit as the heartbeats among a plurality of peaks in which the difference becomes the smallest in waveforms of the differences between the vibration waves before and after delay.

9. The heartbeat detection device according to claim 4, wherein
the second detection unit detects peaks of which the period corresponds to the heartbeats which is detected by the first detection unit and of which the reliability is determined to be high by the determination unit as the heartbeats among a plurality of peaks in which the difference becomes the smallest in waveforms of the differences between the vibration waves before and after delay.
